(19) United States
(12) Patent Application Publication
(10) Pub. No.: US 2014/0235503 A1
(43) Pub. Date: Aug. 21, 2014

(54) PREDICTION METHOD OF GLOMERULAR FILTRATION RATE FROM URINE SAMPLES AFTER KIDNEY TRANSPLANTATION

(71) Applicant: Kyungpook National University Industry-Academic Cooperation Foundation, Daegu (KR)

(72) Inventors: Yong-Lim KIM, Daegu (KR); Young-Ran YOON, Daegu (KR); Jung-Ju SEO, Daegu (KR)

(73) Assignee: KYUNGPOOK NATIONAL UNIVERSITY INDUSTRY-ACADEMIC COOPERATION FOUNDATION, Daegu (KR)

(21) Appl. No.: 13/955,328

(22) Filed: Jul. 31, 2013

(30) Foreign Application Priority Data
Feb. 21, 2013 (KR) 10-2013-0018876

(51) Int. Cl. G01N 33/493

(52) U.S. Cl. CPC G01N 33/493
USPC 506/12; 702/19

(57) ABSTRACT

Disclosed is a prediction method of glomerular filtration rate (GFR) from urine samples after kidney transplantation to provide an information needed for predict renal function after the transplantation, more particularly to a prediction method of glomerular filtration rate (GFR) from urine samples after kidney transplantation, which comprises detecting metabolic profiles of five biomarkers, 5a-androst-3-en-17-one (AS), glycocholic acid (GC), sphingosine (SG), tryptophan (TR) and histidine (HT), from urine samples of patients. Glomerular filtration rate (GFR) after kidney transplantation can be predicted more rapidly and precisely to provide an information needed for predict renal function after the transplantation by using five metabolites as biomarkers. The method provides more specific, sensitive, and reliable biomarkers that monitor clinical outcomes and adverse renal events after kidney transplantation, such as rejection, drug toxicity, delayed graft function, and infection.

### TECHNICAL FIELD

The present invention relates to a prediction method of glomerular filtration rate (GFR) from urine samples after kidney transplantation to provide an information needed for predict renal function after the transplantation, more particularly to a prediction method of glomerular filtration rate (GFR) from urine samples after kidney transplantation, which comprises detecting metabolic profiles of five biomarkers from urine samples of patients.

### BACKGROUND ART

One of the challenges in nephrology today is the limited set of established clinical diagnostic markers that lack sufficient specificity and sensitivity and detect a disease process at a stage when the injury cannot be fully reversed (1). Even estimated GFR using serum creatinine, widely considered the prime putative surrogate endpoint in kidney transplant recipients, has the main limitation that it measures only one aspect of kidney function and does not have high specificity and sensitivity (2-4). Further, if kidney function is not affected, creatinine monitoring will not detect the damage to a graft (5). Therefore, to overcome these limitations and detect kidney dysfunction early, the development of accurate, sensitive, non-invasive, and inexpensive biomarkers would be of considerable value for evaluation of allograft function (5-7).

Renal function, in nephrology, is an indication of the state of the kidney and its role in renal physiology. Glomerular filtration rate (GFR) was known as the best index to reflect the renal function. Most doctors use the plasma concentrations of the waste substances of creatinine and urea (U), as well as electrolytes (E), to determine renal function. These measures are adequate to determine whether a patient is suffering from kidney disease. However, blood urea nitrogen (BUN) and creatinine will not be raised above the normal range until 60% of total kidney function is lost. Hence, the more accurate Glomerular filtration rate should be measured whenever renal disease is suspected, careful dosing of nephrotoxic drugs is required, or renal function is to be predicted after kidney transplantation.

Glomerular filtration rate (GFR) is the volume of fluid filtered from the renal (kidney) glomerular capillaries into the Bowman's capsule per unit time. Glomerular filtration rate (GFR) is equal to the Clearance Rate when any solute is freely filtered and is neither reabsorbed nor secreted by the kidneys. There are several different techniques used to calculate or estimate the glomerular filtration rate (GFR). The GFR can be determined by injecting inulin or the inulin-analog sinistrin into the plasma. Since both, inulin and sinsitrin, are neither reabsorbed nor secreted by the kidney after glomerular filtration, their rate of excretion is directly proportional to the rate of filtration of water and solutes across the glomerular filter. However, the inulin clearance slightly overestimates the glomerular function. In early stage renal disease, the inulin clearance may remain normal due to hyperfiltration in the remaining nephrons. Incomplete urine collection is an important source of error in inulin clearance measurement.

In clinical practice, creatinine clearance or estimates of creatinine clearance based on the serum creatinine level are used to measure GFR. Creatinine is produced naturally by the body (creatinine is a breakdown product of creatine phosphate, which is found in muscle). It is freely filtered by the glomerulus, but also actively secreted by the peritubular capillaries in very small amounts such that creatinine clearance overestimates actual GFR by 10-20%. However, creatinine estimates of GFR have their limitations. All of the estimating equations depend on a prediction of the 24-hour creatinine excretion rate, which is a function of muscle mass. A common mistake made when just looking at serum creatinine is the failure to account for muscle mass. Hence, an older woman with a serum creatinine of 1.4 mg/dL may actually have a moderately severe degree of renal insufficiency, whereas a young muscular male, in particular if black, can have a normal level of renal function at this serum creatinine level. Creatinine-based equations should be used with caution in cachectic patients and patients with cirrhosis.

Recently, advances in high-throughput ‘-omics’ technologies and bioinformatics have led to major discoveries of more specific biomarkers to assess individual disease risk (6). Metabolomics involves measurement of all possible quantitative changes (unbiased) of metabolites (<1 kDa in size) that happen within seconds or minutes after an event and provides a comprehensive picture of the changes in molecular mechanisms that arise from external stimuli (7) or diseases (6, 8, 9). Therefore, this approach offers a specific and sensitive tool for identifying more reliable biomarkers that monitor clinical outcomes and adverse renal events after kidney transplantation, such as rejection, drug toxicity, delayed graft function, and infection (10-14).

Previous metabolomics studies have focused on identifying single individual metabolic biomarkers of a damaged graft, but these metabolites are not specific to monitoring kidney function during transplantation (15-17) and are not yet fully elucidated for their ability to confirm quantitative changes in metabolic profiles associated with kidney function over time. In our previous study (18), we have identified a metabolic phenotype for prediction of pharmacokinetics of immunosuppressive drugs through global urinary metabolic profiling using liquid chromatography-mass spectrometry (LC/MS), which offers greater throughput and coverage of metabolites than previously reported techniques and integrative data analysis. Therefore, here we applied this LC/MS-based global metabolic analysis and our novel integrative approach (FIG. 1) in kidney transplant patients with the aim of identifying a more comprehensive and specific metabolic markers for prediction of kidney function.

### SUMMARY OF INVENTION

The object of the present invention is to provide a method for predicting glomerular filtration rate (GFR) from urine samples after kidney transplantation to provide an information needed for predict renal function after the transplantation, comprising detecting metabolic profiles of 5a-androst-3-en-17-one (AS), glycocholic acid (GC), sphingosine (SG), tryptophan (TR) and histidine (HT) from urine samples of patients.

Further, another object of the present invention is to provide a program storage device readable by a computer, embodying a program for predicting glomerular filtration rate (GFR) from urine samples after kidney transplantation to provide an information needed for predict renal function after the transplantation.

Further, another object of the present invention is to provide a system for predicting glomerular filtration rate (GFR) from urine samples after kidney transplantation to provide an information needed for predict renal function after the transplantation.

Metabolomics can yield markers with improved precision and accuracy for monitoring clinical outcomes. We evaluated the clinical applicability of metabolomics to predict GFR after transplantation using liquid chromatography-mass spectrometry (LC/MS)-based global urinary metabolic profiling along with integrative data analysis and metabolic network analysis. Global metabolic profiling allowed detection of 999 metabolite ions from non-invasive bio-fluid (i.e., urine) samples collected from 12 recipients before (−2 days) and after (5, 30, and 90 days) kidney transplantation. We selected the most important metabolites correlated with GFR using partial least squares (PLS) modeling and mapped them in a metabolic network to understand their role in predicting GFR. Finally, five representative metabolites were selected based on their statistical and biological significance in predicting GFR. We built a clinically applicable prediction equation using identified metabolites. The predicted GFR showed strong correlation with observed GFR (R²=0.71). Moreover, this equation could predict long-term GFR at 2 years after transplantation. In conclusion, we confirmed that urine metabolic phenotype by LC/MS-based metabolic profiling with an integrative approach would be more comprehensive biomarker for predicting early and long-term GFR after kidney transplantation up to 2 years.

Other objects and advantage of the present invention will become apparent from the detailed description to follow taken in conjugation with the appended claims and drawings.

In one aspect of the present invention, there is provided a method for predicting glomerular filtration rate (GFR) from urine samples after kidney transplantation to provide an information needed for predict renal function after the transplantation, comprising detecting metabolic profiles of 5a-androst-3-en-17-one (AS), glycocholic acid (GC), sphingosine (SG), tryptophan (TR) and histidine (HT) from urine samples of patients.

In the method for predicting glomerular filtration rate (GFR) according to the present invention, the phrase “detecting metabolic profiles” means that the quantity (peak intensity) of the five metabolites are detected by LC/MS analysis from the urine samples. Preferably, after the detection of metabolic profiles, an equation for predicting GFR can be derived using PLS loading from PLS analysis between the five metabolites and GFR.

In a preferred embodiment, the method for predicting glomerular filtration rate (GFR) according to the present invention comprises the following steps:

(1) measuring peak intensities of 5a-androst-3-en-17-one (AS), glycocholic acid (GC), sphingosine (SG), tryptophan (TR) and histidine (HT) by using LC/MS analysis from urine samples of patients;

(2) obtaining each normalized metabolite intensities from the peak intensities by performing log₂ normalization using quantile normalization algorithm;

(3) calculating predicted GFR (GFR_{Predicted}) by inserting the normalized metabolite intensities as AS, GC, SG, TR and HT in the following equation 1.

GFR_{Predicted}=0.45HT+0.46AS+0.39TR+0.54SG+0.38GC [Equation 1]

In the method for predicting glomerular filtration rate (GFR) according to the present invention, preferably the equation can predict early and long-term GFR after kidney transplantation up to 2 years.

In another aspect of the present invention, there is provided a program storage device readable by a computer, embodying a program for predicting glomerular filtration rate (GFR) from urine samples after kidney transplantation to provide an information needed for predict renal function after the transplantation, the program comprising the following steps:

(1) being provided peak intensities of 5a-androst-3-en-17-one (AS), glycocholic acid (GC), sphingosine (SG), tryptophan (TR) and histidine (HT) resulted from LC/MS analysis of urine samples of patients;

(2) obtaining each normalized metabolite intensities from the peak intensities by performing log₂ normalization using quantile normalization algorithm;

(3) calculating predicted GFR (GFR_{Predicted}) by inserting the normalized metabolite intensities as AS, GC, SG, TR and HT in the following equation 1.

GFR_{Predicted}=0.45HT+0.46AS+0.39TR+0.54SG+0.38GC [Equation 1]

In the program storage device readable by a computer according to the present invention, preferably the program storage device may be a magnetic recording media, a optical recording media, or a flesh memory card which is readable by a computer.

In another aspect of the present invention, there is provided a system for predicting glomerular filtration rate (GFR) from urine samples after kidney transplantation to provide an information needed for predict renal function after the transplantation, which comprises:

(1) a device for measuring peak intensities of 5a-androst-3-en-17-one (AS), glycocholic acid (GC), sphingosine (SG), tryptophan (TR) and histidine (HT) from urine samples of patients;

(2) a means for inputting the peak intensities;

(3) a data processing device for obtaining each normalized metabolite intensities from the peak intensities by performing log₂ normalization using quantile normalization algorithm and calculating predicted GFR (GFR_{Predicted}) by inserting the normalized metabolite intensities as AS, GC, SG, TR and HT in the following equation 1;

GFR_{Predicted}=0.45HT+0.46AS+0.39TR+0.54SG+0.38GC [Equation 1]

(4) a means for outputting the predicted GFR (GFR_{Predicted}).

In the system for predicting glomerular filtration rate (GFR) according to the present invention, preferably the device for measuring peak intensities may be a LC/MS analysis device, the means for inputting the peak intensities may be connected with the LC/MS analysis device to automatically input the peak intensities, the data processing device may be a computer which can perform the quantile normalization algorithm and the equation 1, and the means for outputting the predicted GFR may be a monitor to display the predicted GFR (GFR_{Predicted}).

Our two main findings are that (1) urinary metabolic profiling using the LC/MS technique with an integrative data analysis provides a more comprehensive and specific metabolic phenotype associated with GFR after kidney transplantation, and (2) a prediction equation derived using this selected metabolic phenotype would be clinically applicable to predict early and long-term GFR (2 years) after kidney transplantation. To the best of our knowledge, our study is the first one regarding integrative metabolomic analysis to elucidate the quantitative changes in metabolic profiles associated with kidney function over time.

We first applied LC/MS-based metabolomic analysis to monitor biochemical changes in urine after kidney transplantation and to assess the quality of graft function before significant pathophysiological damage. Because alterations in kidney function potentially influence several metabolic pathways and concentrations of metabolite in urine, urine is considered an ideal bio-fluid for metabolomic studies in kidney transplantation (37). Therefore, metabolomics using urine can have a significant advantage over other ‘-omics’ for biomarker discovery to monitor graft function. Also, LC/MS techniques have been a contributing factor to identifying a very large portion of urinary metabolomes and quantitative changes of metabolites pre- and post-transplantation.

A single metabolite or individual biomarker may not be specific to kidney function because it may be susceptible to experimental and physiological variations. For instance, changes in nucleoside concentrations or purine metabolism can arise from alterations in kidney function as well as cancer conditions (39). Therefore, we actually need the specific patterns of the biomarker, rather than specific compounds, to monitor medical conditions (40). Because the metabolite network analysis gives more comprehensive biological information of disease markers than individual marker measurements and the PLS model gives differences in the relative contributions of the metabolic pathways represented by five metabolites (18, 41), this integrative approach contributes to finding more accurate and specific metabolites to predict and monitor kidney function relative to a single metabolite or biomarker.

We selected five metabolites for prediction and monitoring of GFR. These metabolites and their associate pathways were enriched after transplantation post-transplant enrichment. These metabolites were tied to specific biochemical pathways such as amino acid and related metabolism (TR and SG), histidine metabolism (HT) and bile acid and steroid metabolism (AS and GC). The tryptophan, which has immune-regulatory function, seemed to increase by the change of normal physiology or pathology of inflammation after transplantation (42). Also, histidine could be increased after transplantation due to its anti-inflammatory and antioxidant effect in impaired kidney (43). In animal study, glycocholic acid was increased according to the recovery of kidney function after acute allograft rejection (44) and sphingosine, known as regulators of kidney physiology, was increased after transplantation, because of it modulate diverse pathways of cell death, including inflammation and immunity (45).

Most of the metabolites from bile acid and steroid metabolism were enriched after transplantation. The concentrations of steroids increased due to the changes of the immune response and enzymes such as cytochrome P450 and AKR1D1(delta4-3-oxosteroid 5beta-reductase), which are essential for production of steroid, after transplantation (18, 29), even though we decreased the dosage of exogenous steroids (e.g., methylprednisolone) in course of time after transplantation. Therefore, in this study, the increased concentrations of steroid and bile acids after transplantation indicated the successful kidney function recovery. Thus, steroid and bile acids can be potentially novel markers for kidney transplant monitoring. We also putatively identified increases in several other metabolites such as hydroxyoctadecadienoic acid, dimethylarginine, L-tyrosine, betaine, guanidinoacetate, guanidinosuccinic acid, homocysteine in pre-transplant samples (data not shown). These markers have been reported as important markers of kidney dysfunction before transplantation (9, 34, 46) but were excluded from our PLS model because of low significance for prediction of GFR (VIP<1.3).

In addition, we confirmed the availability of equation-derived selected metabolic phenotypes to predict early and long-term GFR. Other GFR-estimating equations have been developed and validated at single time points and may lead to error in estimated GFR (47). However, our prediction equation showed good performance over time until 2 years after kidney transplantation. Thus, these urinary metabolic phenotypes could be reasonable and clinically applicable biomarkers for predicting changes in GFR after kidney transplantation.

We could not, however, measure previously reported biomarkers for monitoring graft rejection by nuclear magnetic resonance spectroscopy (NMR) and gas chromatography-mass spectrometry (GC/MS), such as trimethylamine N-oxide (TMAO), betaine, citrates, and amines (13), because of 1) inherent differences in metabolome convergence between other methods and LC/MS that may reveal different classes of metabolites (such as steroids), and 2) using the stable urine samples with normal allograft function. Although our results are based on stable allograft function with no rejections, the selected five metabolites can be used for detection of early graft rejections because they were derived directly from their close correlation and functional association with GFR. Thus, these markers should be more sensitive, accurate, and meaningful in detection of transplant rejections than individual markers. The steps are (1) measurement of the concentrations of the selected five metabolites using conventional assays (48) and simple chromatographic techniques (49, 50) in clinical laboratories and then (2) input into the proposed equation after values are normalized to have zero mean and unit variance. This approach can yield normalized values for GFR to monitor and predict graft rejection as well as kidney function during transplantations.

This invention suggested that urinary metabolic profiling can find more accurate and applicable biomarkers to predict kidney function.

### Advantageous Effects

As described above, the present invention provide a method for predicting glomerular filtration rate (GFR) from urine samples after kidney transplantation more rapidly and precisely to provide an information needed for predict renal function after the transplantation by using five metabolites as biomarkers. These biomarkers are be more sensitive, accurate, and meaningful in detection of transplant rejections than individual markers.

The present invention provides more specific, sensitive, reliable biomarkers that monitor clinical outcomes and adverse renal events after kidney transplantation, such as rejection, drug toxicity, delayed graft function, and infection.

We validated that our integrative approach may contribute to finding accurate and specific biomarkers for predicting and monitoring clinical outcome in kidney transplantation and that this approach can be extended to identify biomarkers of other kidney conditions, such as kidney injury, early or chronic rejection, and drug nephrotoxicity. The selected five metabolites can be used for detection of early graft rejections because they were derived directly from their close correlation and functional association with GFR.

### BRIEF DESCRIPTION OF THE DRAWINGS

The above and other objects, features and other advantages of the present invention will be more clearly understood from the following detailed description taken in conjunction with the accompanying drawings, in which;

FIG. 1 depicts study design and sampling schem after kidney transplantation. First-morning urine samples from 12 patients who underwent kidney transplant were collected pre (−2 day) and post-transplant (5, 30, and 90 days).

FIG. 2 shows pre- and post-transplant changes in serum creatinine concentration (a) and estimated GFR values (b) in 12 patients. a) Plot of mean serum creatinine concentrations (mg/dl) and days after transplantation shows sharp decrease in creatinine concentrations after transplantation (5 day) compared to −2 days pre-transplant and stabilizes on 90 days after transplantation, suggesting recovery of kidney function, b) Plot of mean GFR (ml/min/1.7 m²) and days after transplantation shows sharp increase in GFR after transplantation (5 days) compared to −2 days pre-transplant, and stabilize on 90 days after transplantation, suggesting recovery of kidney function The error bars indicate standard deviation in data.

FIG. 3 illustrates a workflow for metabolomic study. Urine samples were subjected to liquid chromatography-mass spectrometry (LC/MS) analysis to generate global metabolomic profiling data. After data preprocessing and normalization, the mass spectrometric datasets (a pair of m/z and retention time) were used for further multivariate statistical analysis. The partial least squares (PLS) analysis was applied to find the correlation between metabolite profiles (X variable) and estimated GFR (Y variable) and to select key metabolites contributing to the prediction of GRF. These metabolites were then identified by LC/MS/MS analysis and database resources followed by network analyses based on their involved metabolic pathways to understand their functional role in the prediction of kidney function. Finally, a clinically applicable prediction or PLS equation was derived by combining PLS and network analyses to predict the GFR using five key metabolites.

FIG. 4 shows principal component analysis (PCA) score plot (LV1 vs LV2) obtained from patient's urine and pooled quality-control (QC) samples. Black dots indicate patient samples, and red shows pooled QC samples in the same analysis batch. 13 QC samples (5 initial and 8 throughout the sample sequence) analyzed with patient samples. Well-aggregated QC samples indicate that the analysis was valid and that there was no major instrumental bias during LC/MS analysis.

FIGS. 5A to 5D show PLS modeling of urine LC/MS metabolomic data for selection of metabolites highly correlated with GFR. a) PLS score plot for the first two latent variables (LVs) in which each point represents a patient urine sample collected during transplantation, plotted as scores (or coefficients) from the metabolic profile data derived from LC/MS analysis (X variables) and estimated GFR (Y variables). The first LV (LV1) captured the desired variation (arrow) in metabolite profiles during transplantation, which gave three separate groups (circled), while the second LV (LV2) captured inter-individual variations among patients. b) PLS loading plot for the above PLS model in which each point represents a metabolite feature (total 999) detected from LC/MS data and plotted as their respective loadings (regression coefficients) with GFR for PLS LV1 versus loadings from LV2. Black arrow shows a positive relationship with the GFR. Metabolite variables with larger loading coefficient values (positive or negative) have a stronger correlation with the GFR (marked in red boxes; VIP>1.3) and were selected for identification and prediction of GFR. The blue upward arrow shows metabolites with a positive correlation while red downward arrow shows negative correlation with GFR. c) A partial listing of the identified significant metabolites with their scaled and centered values of coefficient and error bars indicating jack-knife uncertainties (51). d) Internal validation of this PLS model by 20 permutation tests to check accidental correlations and data over-fitting shows that all R² (goodness of fit) and Q² (predictability of model) values from the permuted models (left) are smaller than those of the original model (far right) and also the negative intercept (−0.05) for Q2 at the Y-axis. This collectively indicates the validity of the PLS model.

FIG. 6 shows cross-validation plot for predicted GFR versus observed GFR values by the PLS model using all 999 metabolite variables. Cross-validation was done by exclusion of 1/7^{th} of the dataset and predicting the excluded data as the external sample set. This approach validated the PLS model for prediction of external samples.

FIG. 7 is an internal validation plot for final PLS equation showing that the five-metabolite PLS equation is still valid for prediction of GFR as all permuted R² and Q² values (far left) fall below that of the original model (far right) and the intercept is at −0.136 at the Y-axis.

FIG. 8 shows metabolic network for 25 identified metabolites. Each node represents a metabolite, and size of a node is proportional to its degree of correlation with GFR. The borders of nodes are colored based on positive (blue) and negative (red) correlation with GFR. The connecting edges show metabolic direct reactions (solid line) or possible reactions (dotted arrow) with several intermediates; involved enzymes are named on solid edges. This metabolic network can be distinguished in three major metabolism modules, shown in different colors of node. All abbreviations used for enzymes or genes and pathways/reactions are from KEGG identifiers (http://www.genome.jp/kegg/kegg3.html).

FIGS. 9A and 9B depict plots for change in normalized intensities of significant metabolites during transplantation. HT, histidine; AP, acetylspermidine; TR, tryptophan; AS, 5a-androst-3-en-17one; AM, adenosyl methionine; DC, 3a,7a-Dihydroxy-5b-cholestane; GC, Glychocholic acid; SG, sphingosine; PG, Phenylglycine; KA, Kynuerenic acid; PC, Phosphocreatine; CN, Camosine; DG, Dimethylguanosine. a) Overlay plot of normalized intensity changes with days after transplantation for some of the identified metabolites, which are positively correlated with GFR (shown by blue arrow in FIG. 5b). b) Overlay plot of normalized intensity changes with days after transplantation for the identified metabolites, which are negatively correlated with GFR (shown by red arrow in FIG. 5b).

FIGS. 10A and 10B depict scatterplots for the predicted GFR (GFR_{Predicted}) from PLS equation using the selected five metabolites versus the observed GTF for 90 days (a) and until 2 year (b) after transplantation. (a) This prediction equation with only five metabolite concentrations (GFRₙₒᵣₘ=0.4HT+0.46AS+0.39TR+0.54SG+0.38GC) predicts GFR well (R²=0.7066) for 90 days after transplantation, included in model. (b) This equation was also found suitable (R²=0.5031) for the prediction of GFR values until 2 years, which were not included in the model initially. AS=5a-androst-3-en-17-one; GC=glycocholic acid; SG=sphingosine; TR=tryptophan; HT=histidine.

### MODE FOR INVENTION

Practical and presently preferred embodiments of the present invention are illustrated as shown in the following Examples. However, it will be appreciated that those skilled in the art, on consideration of this disclosure, may make modifications and improvements within the spirit and scope of the present invention.

Materials and Methods

1) Study Design and Eligibility of Patients

In this prospective and exploratory study, we included 12 patients who underwent a first kidney transplantation and gave an informed consent before enrollment. We excluded patients who were under age 18 years and/or who had clinically significant events within 3 months prior to transplantation, such as cardiac congestive failure, hepatic failure, neoplastic or infectious disease, cardiovascular events, multi-organ transplantation, or anticipated graft failure to transplantation. All patients were on triple immunosuppressive therapy, including tacrolimus, corticosteroids, and mycophenolate mofetil or mizoribine according to local practice during the study. The protocol (KNUH_{—}06-0013) was approved by the Ethics Committees of Kyungpook National University Hospital.

2) LC/MS-Based Global Metabolic Profiling

To analyze global (non-targeted or unbiased) metabolic profiles, the LC/MS-based analysis was performed in the following steps: urine sample preparation, full scan (50-900 m/z; ratio of mass to charge) LC/MS analysis, data preprocessing, peak detection and alignment, peak intensity normalization, and export annotated peak data table production for further multivariate statistical analysis. The LC/MS analysis system consisted of an Alliance HPLC (Waters) connected to a linear ion-trap mass spectrometer (LXQ; Thermo Electron, Waltham, Mass.).

3) Urine Sample Preparation

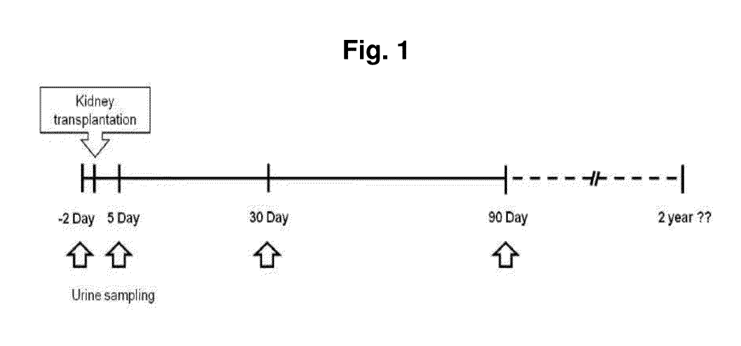

First-morning urine samples for metabolic profiling were collected in clean amber-colored sterilized tubes before (−2 day) and after (5, 30, and 90 days) kidney transplantation and prior to administration of immunosuppressants (FIG. 1). Each urine sample of 200 μl was diluted in 400 μl of acetonitrile (Merck, Darmstadt, Germany) containing bromophenylalanine, tubercidin, and amitriptyline as internal standards (Sigma-Aldrich, St. Louis, Mo., USA, or Steraloids Inc.) for protein precipitation. Urine samples were then centrifuged at 2000 rpm for 5 min to remove particulate matter after incubating at 4° C. for 30 min. The supernatants were used for further LC/MS analysis.

4) LC/MS Analysis

The 5 μl of the prepared samples was injected into the LC/MS system. The separation of urine metabolites was accomplished using a Kinetex C₁₈ (100×2.1 mm i.d., 2.6 μm particle size) column (Phenomenex, Torrance, Calif., USA) maintained at 40° C. The mobile phase contained aqueous 0.1% formic acid (A) and 0.1% formic acid in acetonitrile (B), with a gradient starting at 10% B for 3 min, which was increased to 80% B over 12 min, further increased to 90% B for 25 min, and held constant for 4 min. The system was re-equilibrated to the starting conditions of 10% B for 6.5 min, making the total runtime 35 min. The mass spectrometer was used in full-scan positive-ion mode in the range of 50-900 m/z with an electro-spray ionization voltage of 5 kV, capillary temperature 280° C., sheath gas flow 20 units, tube lens 120 V, and capillary voltage 35 V. To avoid systematic variation, all samples were randomized prior to LC/MS analyses and quality-control (QC) samples, together with internal standards in all samples were incorporated throughout the analysis (19, 20).

5) Data Preprocessing, Peak Detection and Alignment

To convert chromatographic information of metabolites into a dataset suitable for multivariate statistical analysis, data preprocessing war performed including background subtractions, peak filtering and detection, and peak alignment using XCMS software version 1.22.1 (21). This program converts three-dimensional LC/MS data (m/z, retention time (T), and ion intensity) to a readily accessible two-dimensional data matrix (a pair of m/z and T). The steps for this process, using the default XCMS parameters (http://www.bioconductor.org/packages/release/bioc/html/xcms.html), are as follows: 0.1 m/z for peak picking, peak-width at half-maximum of 30 s, and peak group bandwidth of 10; the signal-to-noise ratio threshold was set to 10. After peak detection and de-convolution by XCMS, each detected metabolic peak was identified according to its m/z and T. The blank chromatograms were used for background subtraction from sample chromatograms using the Xcalibur software utility (Thermo Electron). Files were then converted to NetCDF format using the Xcalibur file-converting utility.

6) Data Normalization and Imputation

The measured peak intensities were normalized to the log 2 intensities using the quantile normalization algorithm of the preprocessCore package (22) with R language (version 2.11.1) to remove systematic variation in peak intensities. Then, the normalized peak intensities were exported to CSV files to produce the annotated peak data table along with unique peak identifiers. We excluded the peaks with missing values of more than 50% because of a low abundance of those metabolic ions, below the limit of detection, with an imputed minimum peak area observed for that metabolite ion. Finally, the normalized peak intensities with their pairs of m/z and T were used for further statistical analysis. The three internal standards were spiked in all samples to correct any variation. The overall T and peak area variations (coefficient of variance, CV) were not more than 20%. The peaks with unacceptable variations in QC samples (CV>20%) and the peaks corresponding to m/z of drugs or drug metabolites were excluded from exported peak data.

7) Multivariate Data Analysis

To find the most important metabolite correlated with kidney function and contributing to that prediction, we applied a supervised multivariate analysis, the partial least squares (PLS) model using the SIMCA P+ software (version 12; Umetrics, Uppsala, Sweden). PLS model (23) was validated to verify its ability to predict by 1) cross-validation using the leave-one-out approach, 2) internal validation applying 20 permutation tests, and 3) comparison of the goodness of fit (R²) and predictive ability (Q²).

8) PLS Model

The PLS model reduces the dimensionality of multivariate X variable and observes the linear combination of relationship (latent variables, LVs) between X variable (metabolite profiles) and Y variable (GFR) to predict GFR. Therefore, the PLS analysis produced coefficients for each LV by a linear combination of metabolite variables that define PLS scores for samples and regression coefficients (PLS loadings) for metabolites (24). The first two LVs were determined based on high predictive ability and low data variation, which explained their cross-validation Q² values and eigenvalues, respectively. After the PLS modeling, the variable influence on projection (VIP) was essentially examined to measure and identify which specific metabolites contributed most to GFR or a trend observed in the data. We used VIP values to select metabolic profiles most related to GFR and most contributing to predict GFR (VIP>1.3) in the PLS model.

9) Metabolic Identification

To select significant metabolites (VIP>1.3), we applied LC/MS/MS-based method (tandem mass spectrometry) to acquire MS/MS spectra for respective m/z and T (Table 1). Then, we identified the potential metabolites by matching MS/MS spectrum of metabolic ions to a reference metabolite database, such as HMDB (25), Metlin (26), and LIPID MAPS (27), and relevant literature. To confirm the results of metabolite identification, we compared selected metabolite ions and the commercial standards of these potential metabolites. If the commercial standards were not available easily, putative identification was done. In the cases of conjugated steroids, glycerophospholipids, and conjugated nucleosides, predictive fragmentation patterns and T were also considered to identify their chemical class rather than exact names. Finally, we confirmed the chemical structure from obtained MS/MS data through possible fragmentation reaction mechanism. Because of some inherent limitations of the MS technique and limited availability of metabolomics resources, we were unable to name all selected metabolites.

10) Metabolic Network Analysis

Following identification of metabolites, we drew the metabolic network using Cytoscape (version 2.6.2) and the KEGG (28), HMDB (25), and MetaCyc pathway databases (29) and other literature (30). All the abbreviations used for enzymes or genes and reactions were from KEGG identifiers (http://www.genome.jp/kegg/kegg3.html).

### EXAMPLE 1

### Patient Characteristics

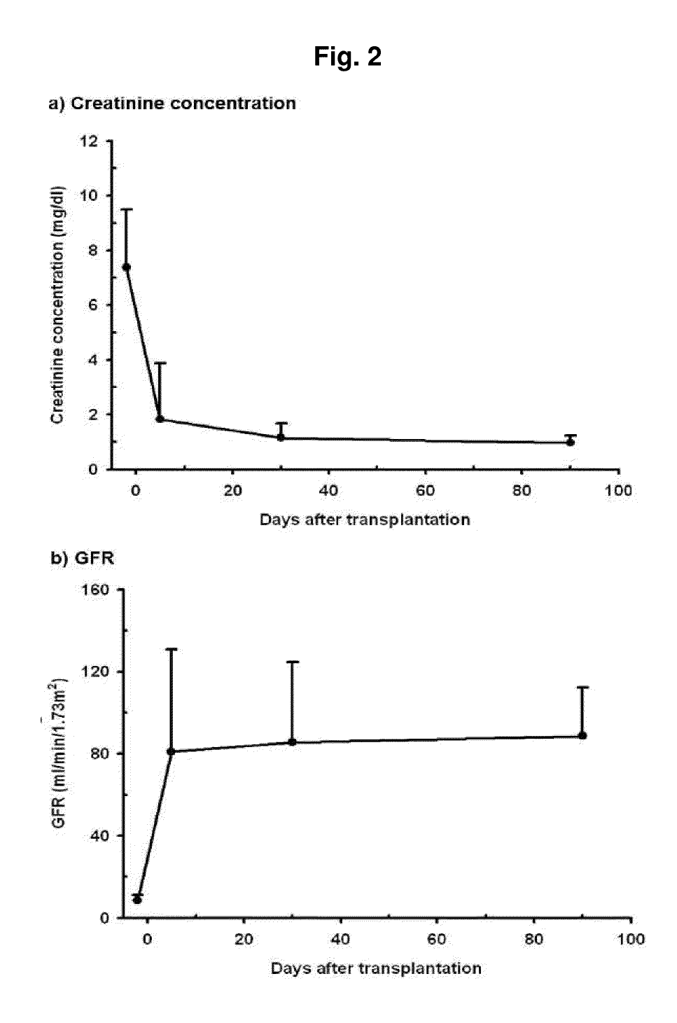

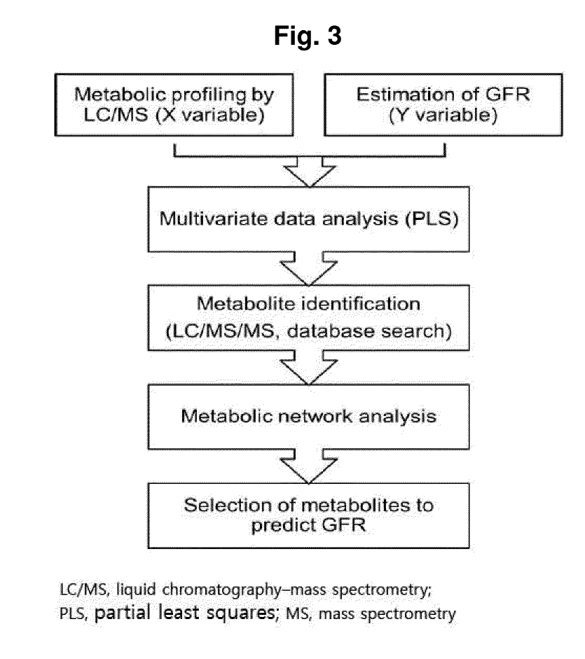

Twelve patients with a mean age of 42±10.4 years were enrolled and followed up until 90 days after transplantation. A total of 66.7% of them had chronic glomerulonephritis. No acute rejections occurred up to 90 days. The estimated GFR by creatinine level showed a sharp increase from 8.6±2.57 to 80.9±49.86 and stabilized at 88.7±23.82 ml/min/1.73 m² on 90 days after transplantation (FIG. 2). Because GFR describes kidney function best (31), it was selected as a response variable (Y) in the PLS analysis. There was also a significant increase in urine volume (average 1354.8±578.2 ml) on 5 days, and then a stable mean urine output of 870.0±445.98 ml was observed on 90 days after transplantation (data not shown).

### EXAMPLE 2

### Urinary Metabolic Profiling Pre- and Post-Kidney Transplantation

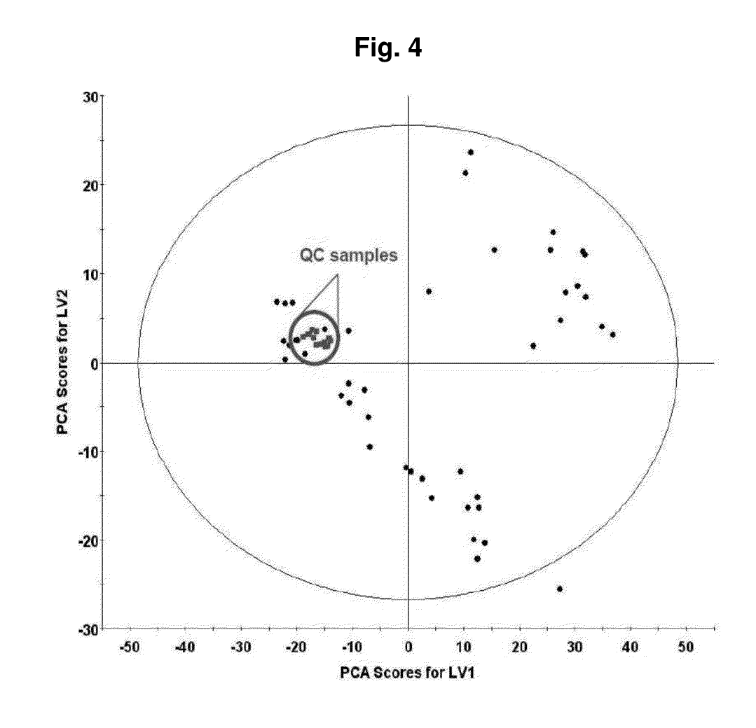

We obtained global metabolic profiles of urine samples that were collected pre and post-transplantation from 12 kidney recipients (FIG. 1). Each peak-intensity represents the individual urinary metabolomes. And chromatograms of urinary metabolic profiling showed significant differences in peaks depending on the treatment time even after exclusion of background and exogenous (drug) peaks (data not shown). After exclusion of peaks corresponding to drugs and drug metabolites, 999 common metabolite ions (metabolite features) from endogenous metabolites with their respective intensities (peak areas) were obtained as a metabolic dataset. These intensities were used as a predictor variable (X) during subsequent multivariate data analysis. The principle component analysis (PCA) plot of quality-control values indicated that the stability and reproducibility of the LC/MS profiling analysis was confirmed that obtained metabolite concentration changes were caused by biological conditions of patients, not by experimental or instrumental variation (FIG. 4).

### EXAMPLE 3

### Selection of Metabolites Highly Correlated with GFR

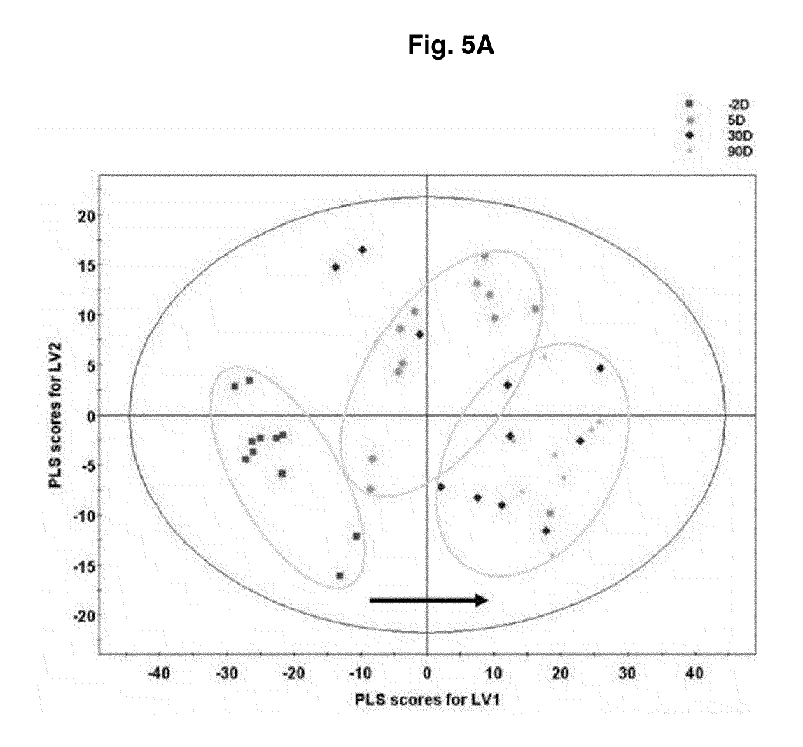

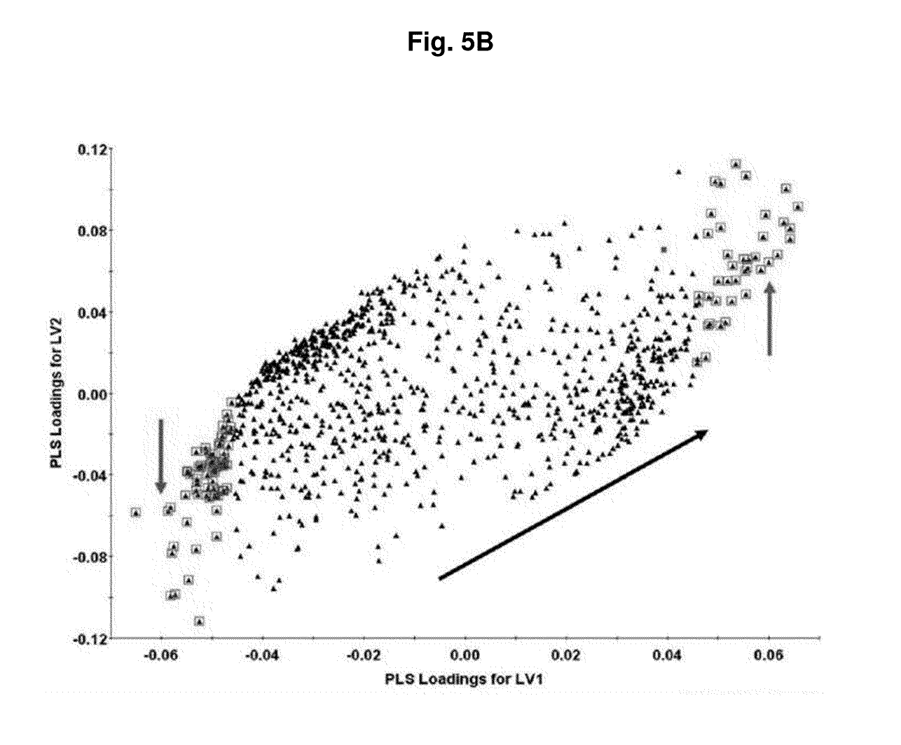

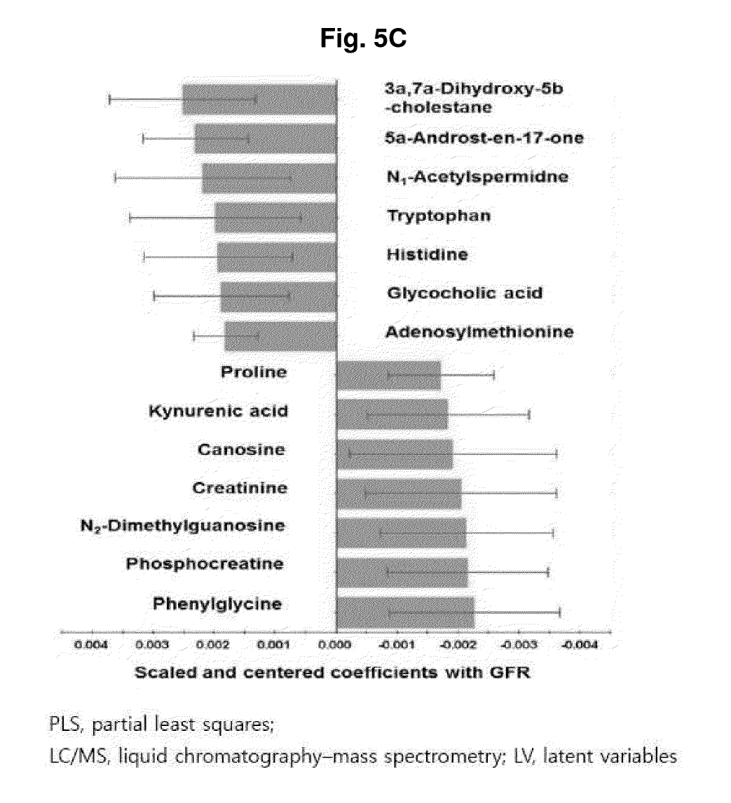

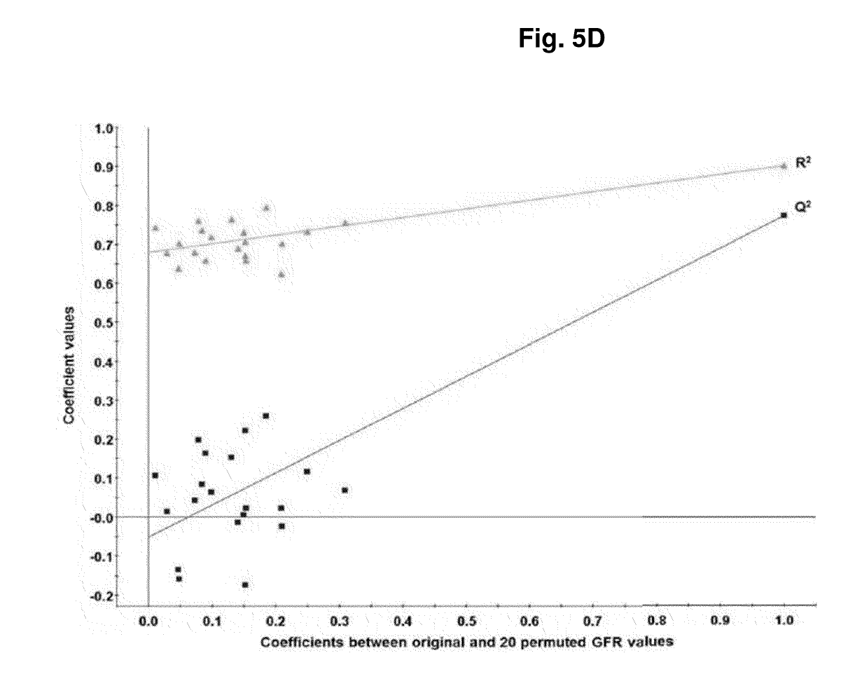

We applied the PLS model to select potential metabolites that had the highest correlation with GFR. First, we selected two statistically significant LVs (LV1 and LV2) from PLS analysis as indicated by the high goodness of fit (R²=0.82) and high eigenvalues (15.8 and 3.83 for LVs, respectively). The plot of PLS scores from two LVs shows three groups (circled) among patient samples (FIG. 5a). The trajectory of plots indicated by the arrow reflects the significant changes in metabolic profiles, which correlated well with GFR over time. The metabolic changes between pre- and post-transplantation were obvious, but the metabolic pattern overlapped among samples that were collected on 30 and 90 days. This pattern showed that its differentiation was decreased due to the stable kidney function after kidney transplantation. This PLS model had a high predictability (Q²=0.702) and explained overall 81.9% of the total variation. LV2 explained inter-individual variation in metabolite profiles (35.6%), and LV1 represented metabolic changes during transplantation, as indicated by the arrow in FIG. 5a (46.3%). In FIG. 5b, the metabolites with high coefficient values for LV1 were strongly associated with GFR and contributed to predict GFR. The blue upward arrow indicates metabolites with a direct or positive correlation with the GFR, and the red downward arrow indicates an inverse or negative correlation.

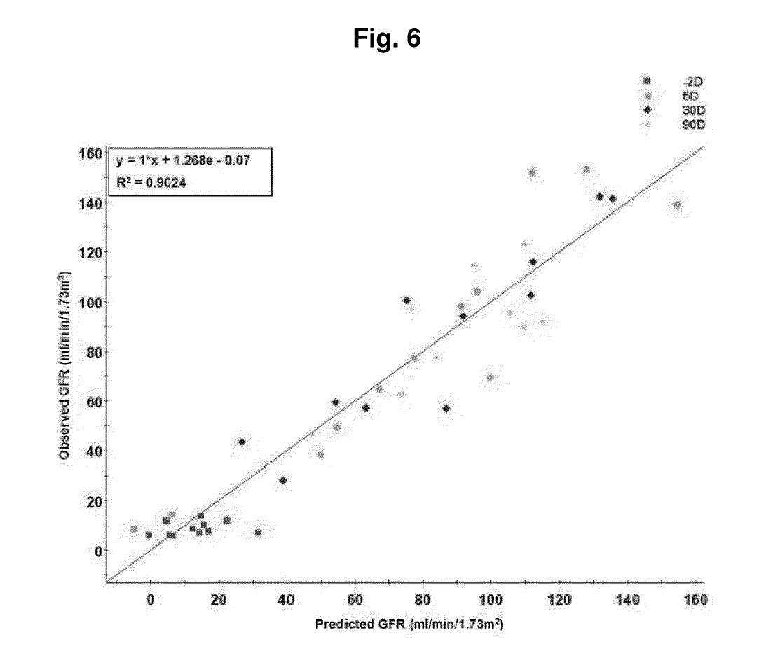

To identify the most significant metabolites, we used their VIP values from LV1, which reflect the relative importance of each metabolite in predicting GFR. The 111 selected metabolite variables (VIP>1.3) are enclosed in red boxes in FIG. 5b. Some of these metabolites displayed positive correlations with GFR that indicated an increase in metabolite level after kidney transplantation, whereas others had negative correlations indicating the opposite (FIG. 6). FIG. 5c gives a partial listing of the identified significant metabolites with their coefficient values with GFR. Some VIP values showed higher confidence intervals (FIG. 5c and FIG. 6), suggesting that their possible contribution to the PLS model might be due to experimental variations, and such metabolic features were excluded from subsequent analysis.

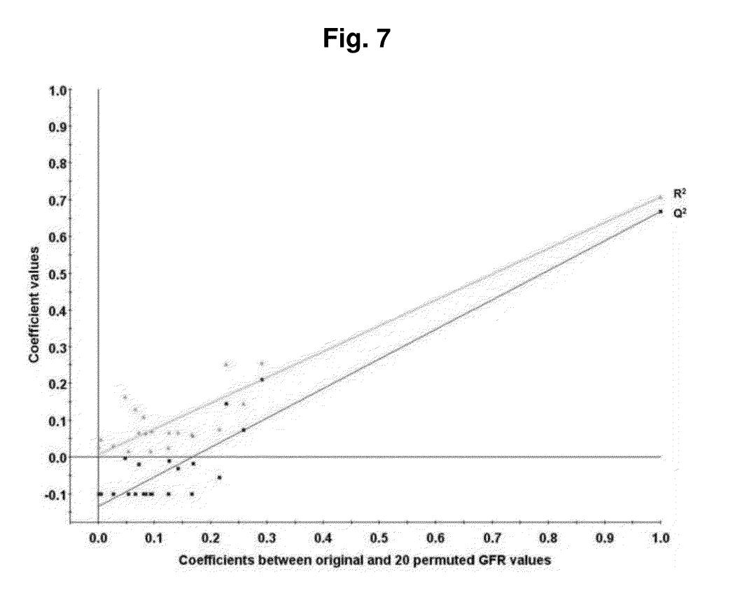

We validated our PLS model by internal validation with 20 random permutation tests (32) and seven rounds of cross-validation (24). As shown in FIG. 5d, the goodness of fit (R²) and predictability (Q²) of permuted models (far left) were much smaller than our original model (far right). Also, the negative intercept at −0.05 indicates that there was no over-fitting of data in this model. The cross-validation by exclusion of 1/7^{th} of the dataset and predicting the excluded samples with the new model gave a root mean square error of prediction (24) for GFR of 15.5 ml/min/1.73 m² (FIG. 7). All predicted GFR values using these 111 selected metabolites showed good correspondence with actual measured GFR (R²=0.819, data not shown). These results assured the validity of our PLS model and selected metabolite markers for predicting GFR.

### EXAMPLE 4

### Identification of Key Metabolites Correlated with GFR

For chemical identification of the selected 111 metabolites (VIP>1.3), the LC/MS/MS-based method was applied. The 15 key metabolites among 25 metabolites (Table 1) with their associated metabolic pathways are summarized in Table 2 listed in descending order of the VIP.

TABLE 1

| | | m/z | Retention |
|---|---|---|---|
| Metabolite | HMDB ID | (MS→S/MS) | time (min) |
| Creatinine* | HMDB00562 | ^{†}114, 115 →86 | 1.3 |
| L-Proline* | HMDB00162 | 116 →69, 89 | 1.4 |
| Guanidinoacetate | HMDB00128 | 118 | 1.3 |
| Creatine* | HMDB00064 | 132 →111, 90 | 1.4 |
| L-Lysine* | HMDB00182 | ^{†}146, 147 →129, 127, 100 | 1.3 |
| 2-Phenylglycine* | HMDB02210 | 152 →135, 106 | 1.3 |
| L-Histidine* | HMDB00177 | ^{†}156, 157→110 | 1.2 |
| L-Arginine* | HMDB00517 | 175 →157, 130 | 1.2 |
| Citrulline* | HMDB00904 | 176 →158, 116 | 2 |
| N1-Acetylspermidine | HMDB01276 | 188 →117, 130, 171, 146 | 1.2 |
| Kynurenic acid* | HMDB00715 | 190 →172, 162 | 1.4 |
| L-Tryptophan* | HMDB00929 | 205 →188 | 2.1 |
| Phosphocreatine | HMDB01511 | 212 →132 | 1.4 |
| Carnosine* | HMDB00033 | 227 →210, 180 | 1.3 |
| Deoxycytidine* | HMDB00014 | 228 →112 | 1.3 |
| Cytidine* | HMDB00089 | 244 →112 | 1.4 |
| 5a-Androst-3-en-17-one | HMDB06046 | ^{†}273, 274 →257 | 16.5 |
| Androstenol* | HMDB05935 | 275 →257 | 12.8 |
| Guanosine* | HMDB00133 | 284 →152 | 2.7 |
| Adrenosterone* | HMDB06772 | 301 →241, 139, 268, 283 | 13.3 |
| N2,N2-Dimethylguanosine* | HMDB04824 | 312 →180 | 1.5 |
| S-Adenosylmethionine | HMDB01185 | 400 | 13.1 |
| 3a,7a-Dihydroxy-5b-cholestane* | HMDB06893 | 404.5, 405 →387, 309, 285, 267 | 16.6 |
| Glycocholic acid* | HMDB00138 | ^{†}466 →430, 412, 448 | 14.4 |
| Sphingosine | HMDB00252 | 300 →241, 282 | 9.7 |

m/z, ratio mass to charge; MS, mass spectrometry;
*identified using authentic standards and others were putatively identified
^{†}m/z from isotopepatterns is included HMDB, Human metabolomedatabase

TABLE 2

| | | | Pathway/reaction | |
|---|---|---|---|---|
| Metabolite name | HMDB ID | VIP | or class | Reference |
| 3a,7a-Dihydroxy- | HMDB06893 | 2.1 | Bile acid biosynthesis | KEGG pathway |
| 5b-cholestane | | | | (ko00 120, 121) |
| 5a-Androst-3-en-17-one | HMDB06046 | 1.9 | Steroids and steroid | HMDB class |
| | | | derivatives | |
| Phenylglycine | HMDB02210 | 1.8 | Glycine, serine, and | KEGG pathway |
| | | | threonine metabolism | (map00260) |
| Creatinine | HMDB00562 | 1.7 | Creatine and creatinine | Ref 34 and KEGG |
| | | | metabolism | pathway (ko00330) |
| N1-Acetylspermidine | HMDB01276 | 1.7 | Spermineand | MetaCyc and |
| | | | spermidine degradation | KEGGpathway |
| | | | | (ko00330) |
| N2-Dimethylguanosine | HMDB04824 | 1.7 | Purine metabolism | KEGGpathway |
| | | | | (ko00230) |
| Phosphocreatine | HMDB01511 | 1.7 | Creatine and creatinine | Ref 34 and KEGG |
| | | | metabolism | pathway (ko00330) |
| L-Histidine | HMDB00177 | 1.6 | Histidine metabolism | KEGG pathway |
| | | | | (ko00340) |
| L-Tryptophan | HMDB00929 | 1.6 | Glycine, serine, and | KEGG pathway |
| | | | threoninemetabolism | (map00260) |
| Carnosine | HMDB00033 | 1.5 | Histidine metabolism | KEGG pathway |
| | | | | (ko00340) |
| S-Adenosylmethionine* | HMDB01185 | 1.5 | Arginine and proline | KEGG pathway |
| | | | metabolism | (ko00330) |
| Glycocholic acid | HMDB00138 | 1.5 | Bile acid biosynthesis | KEGG pathway |
| | | | | (ko00120, 121) |
| Kynurenic acid | HMDB00715 | 1.5 | Tryptophan metabolism | KEGG pathway |
| | | | | (ko00380) |
| L-Proline | HMDB00162 | 1.5 | Arginine and proline | KEGG pathway |
| | | | metabolism | (ko00330) |
| Sphingosine | HMDB00252 | 1.3 | Sphingolipid metabolism | KEGGpathway |
| | | | | (ko00330) |

VIP, variable influence on projection; KEGG, Kyoto Encyclopedia of Genes and Genomes, All abbreviations used for pathways and reactions are from KEGG identifiers (http://genome.jp/keg/kegg3.html)

### EXAMPLE 5

### Metabolic Network Analysis of the Identified Metabolites

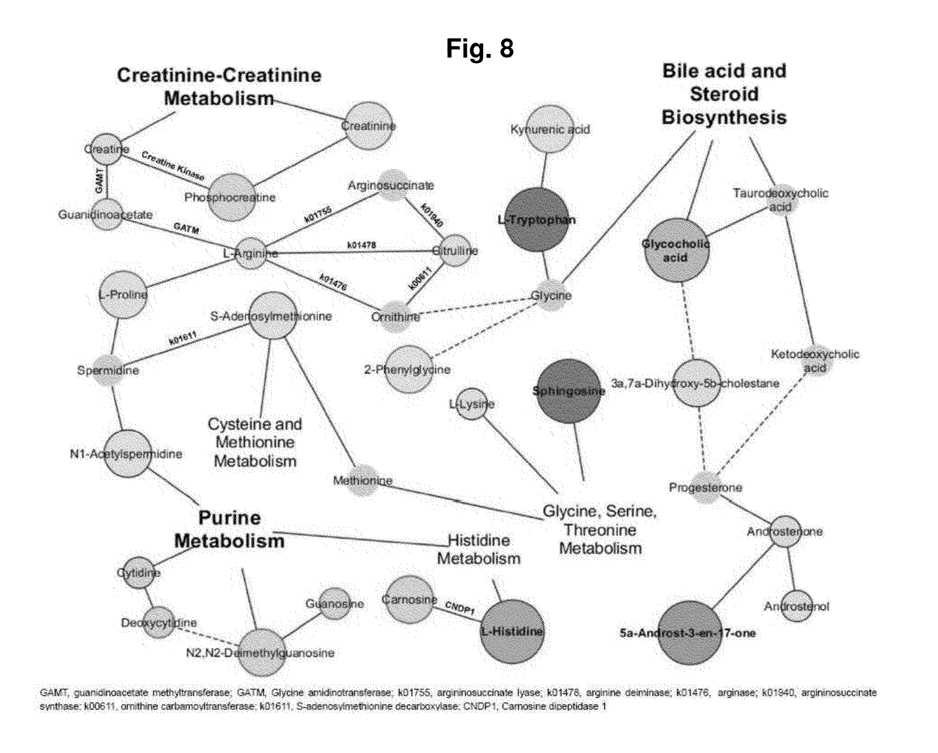

To understand biochemical correlations among these differentiating metabolites and their functional role in predicting kidney function, we constructed a hypothetical metabolic network using the 15 identified metabolites and their neighbors from the metabolic interactome, based on the associated metabolic pathways obtained from databases (FIG. 8). This network showed major network modules and representing metabolism related to 1) creatinine-carnitine (33), 2) the urea cycle and other amino acids, and 3) nucleoside/purine and steroid or bile acid. This result suggested that these metabolites and/or enzymes or genes in a major metabolic pathway may have a significant functional association with kidney function after transplantations. Some metabolites in the network from creatine-creatinine, tryptophan, glycine, purine, cholesterol, and bile acid metabolism were already known to play role in kidney function (9, 11, 14, 34-36). Also, some enzymes involved in networks such as CNDP1, creatinine kinase, and some urea cycle enzymes were known to be associated with kidney function/dysfunction (37, 38). However, several metabolites in the network have never been reported as markers of kidney function, and they may be novel markers to monitor kidney function during transplantation.

### EXAMPLE 6

### Prospective Changes of Identified Metabolites According to the GFR During Transplantation

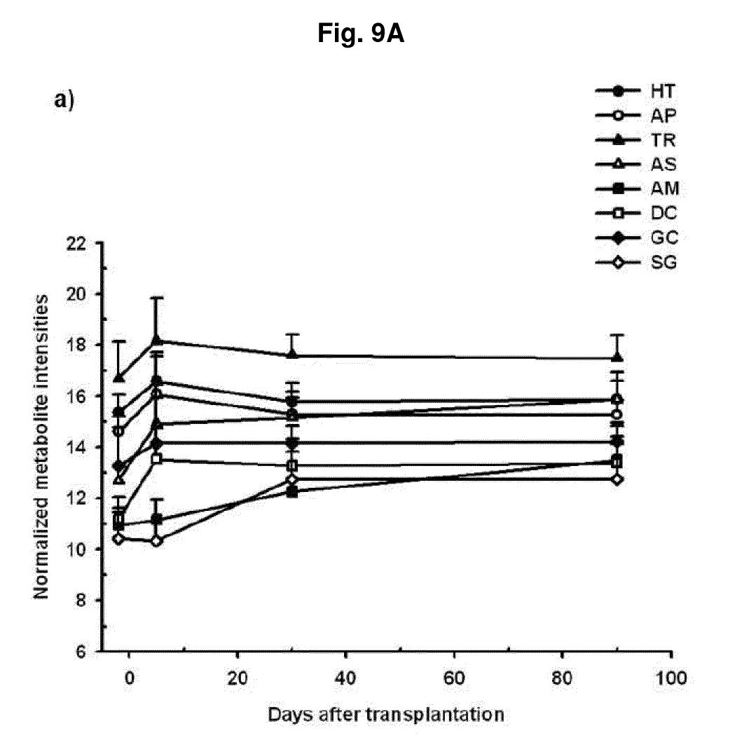

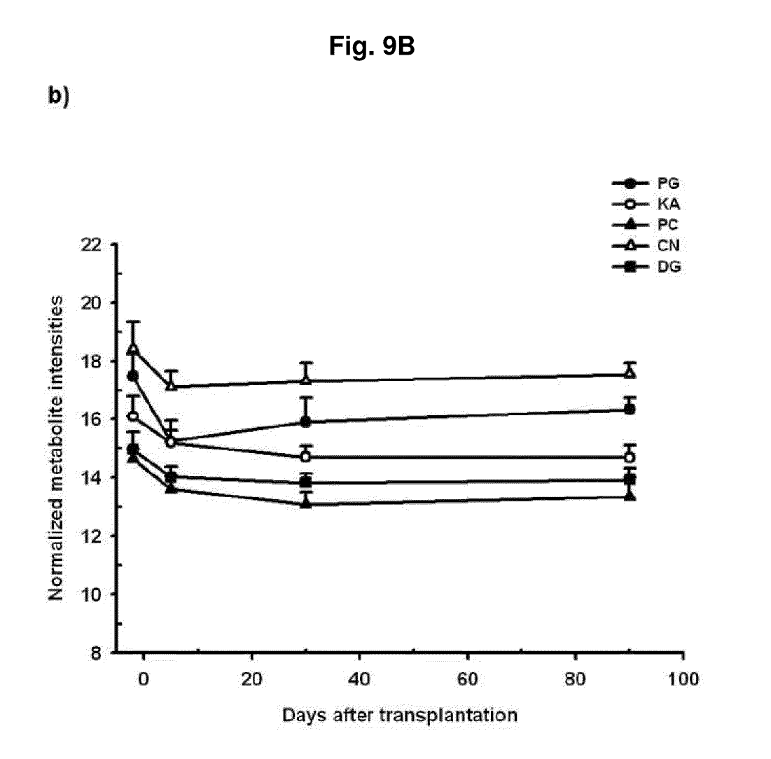

The normalized intensities of 15 identified metabolites were changed in process of treatment time. Some of these metabolites, which were positively correlated with the GFR, elevated drastically at 5 days after transplantation, followed by slightly decreased and stable levels at 30 and 90 days (FIG. 9a). Also, these metabolites were shown as blue edge nodes in metabolic network (FIG. 8). While other metabolites, which were negatively correlated with GFR, considerably decreased at 5 days and gradually increased and stabilized up to 90 days after transplantation (FIG. 5b), shown as red edge nodes in FIG. 8. These patterns suggested that the identified metabolites and their associated metabolic pathways were expected the increase or decrease after transplantation, according to positive or negative relationship with GRF and recovery of GFR after transplantation. In the major metabolic network, the bile acid and steroid metabolism showed remarkable increase after transplantation. However, the creatine-creatinine pathway and urea cycle and purine metabolism were expected to decrease after transplantation due to their inverse relationship with GFR.

Based on these findings, we suggest that these 15 metabolites, such as 3a,7a-dihydroxy-5b-cholestane, Glycocholic acid, phenylglycine, tryptophan, and N2-dimethylguanosine, can be equal to or even better than single urinary metabolites as predictive markers of kidney function because of 1) higher correlation coefficients with GFR, 2) being more representative of a major metabolic pathway than creatinine (Table 1), and 3) changing according to the recovery of kidney function during transplantation.

### EXAMPLE 7

### Clinically Applicable Metabolic Phenotype to Predict GFR

All 15 identified metabolites were still not convenient to predict GFR in actual clinical practice. Therefore, we applied an integrative approach reported previously (18) and selected a metabolic phenotype based on 1) coefficients and predictive ability for GFR (i.e., VIP) from the PLS model; 2) being representative of three major modules in the metabolic network; and 3) being easy to measure in clinical laboratories. Finally, five metabolites, i.e., 5a-androst-3-en-17-one (AS; VIP=1.9), glycocholic acid (GC; VIP=1.5), sphingosine (SG; VIP=1.3), tryptophan (TR; VIP=1.6), and histidine (HT; VIP=1.6) were selected. These five metabolites represent the following metabolic pathways: amino acid and related metabolism (TR and SG), histidine metabolism, and bile acid and steroid metabolism (AS and GC). That is, these five metabolites represented the major network modules with their associated pathways and could be used to improve the prediction of GFR.

### EXAMPLE 8

### Derivation of New Prediction Equations for GFR Based on the Urine Metabolites

The prediction equation for GFR was derived using PLS loading from PLS analysis between five selected metabolites and GFR, as follows:

(1) analysing urine metabolites by using LC/MS analysis: 999 common metabolite ions (metabolite features, three-dimensional data, unit: m/z, retention time (T), and ion intensity);

(2) transforming the resulting data of LC/MS analysis into an analyzable form (Data preprocessing by XCMS software version 1.22.1). XCMS. Performing non-linear retention time alignment, feature detection and feature matching by using a program (http://metlin.scripps.edu/xcms/index.php). As a result, three dimensional data (m/z, retention time, and ion intensity) was transformed into two dimensional data (unit: a pair of m/z and T and ion intensity). 999 metabolites were used as standard (Anal. Chem. 2006, 78, 779-787). Then, in order to remove systematic variation, the measured peak intensities were log₂ normalized by using R software to provide normalized metabolite intensities;

(3) selecting metabolites which have highest correlation with GFR by PLS analysis using SIMCA P+ software (version 12; Umetrics, Uppsala, Sweden). PLS (the partial least squares) is one of models used to reduce dimensionality of X variable and repeats regression analyses to find GFR_{predicted} to minimize prediction error using Y variable (GFR in the present invention) of which value was known. In the present invention, the values of 999 metabolite before (−2 days) and after (5, 30, and 90 days) kidney transplantation was applied as X variable and the GFR values of the corresponding time was applied as Y variable. PLS modeling was performed using SIMCA software. The metabolites which have highest correlation with Y were selected via VIP (variable importance in the projection) value. VIP value is a value which calculating the correlation of all X term (Xk) into Y in the model. SIMCA software can automatically calculate the VIP value and correlation coefficients values. Among the total urine metabolites, 111 metabolites were selected which have highest correlation with GFR (selection criteria: VIP>1.3).

(4) selecting 15 metabolites among the selected 111 metabolites by using LC/MS/MS-based method, metabolites database (human metabolome database (HDMB), metabolite and tandem MS data base (METLIN), lipid metabolite and pathways strategy (LIPID MAPS), and relevant literature;

(5) selecting 5 metabolites among the selected 15 metabolites, which have metabolic pathway relevant to renal function via metabolites network analysis, which have high correlation with GFR using integrative approach, which can replace creatinine as a biomarker of renal function, and which have distinct variation before and after transplantation;

(6) in order to confirm the predictivity of the selected 5 metabolites for renal function, inserting 5 metabolites as X variable and GFR as Y variable and performing PLS modeling as above to provide correlation coefficient. As a result, the following prediction equation for GFR was derived:

GFR_{Predicted}=0.45HT+0.46AS+0.39TR+0.54SG+0.38GC

(7) inserting normalized metabolite intensities as AS, GC, SG, TR and HT in the above equation to calculate the predicted GFR (GFR_{Predicted}).

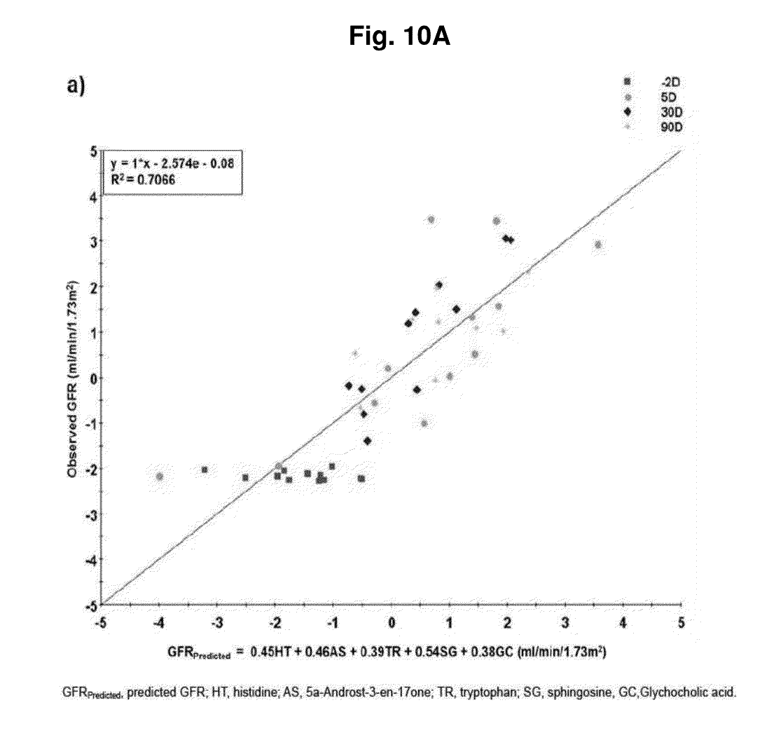

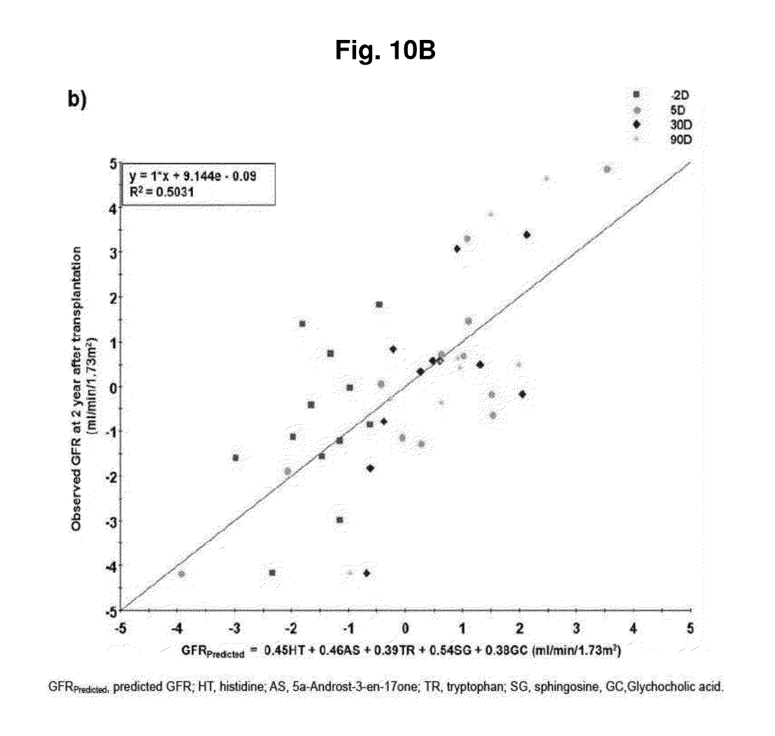

The GFR_{Predicted} for all samples showed excellent correlation with observed GFR (R²=0.71; FIG. 10a). The new PLS model with only these five metabolites had similar predictive ability (Q²=0.67) as that of the original model with all 999 metabolites. The internal validation of this model was found acceptable (FIG. 7). Also, the further predictability of this equation was confirmed by the fact that the GFR_{Predicted} at 2 years showed acceptable correlation with observed GFR (R²=0.50, FIG. 10b). These results suggested that this prediction equation was suitable for prediction of early and long-term GFR after kidney transplantation.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

### REFERENCES

1. American Society of Nephrology Renal Research Report. J Am Soc Nephrol 2005; 16(7):1886-1903.

2. Stevens L A, Levey A S. Measurement of kidney function. Med Clin North Am 2005; 89(3):457-473.

3. Filler G, Browne R, Seikaly M G. Glomerular filtration rate as a putative ‘surrogate end-point’ for renal transplant clinical trials in children. Pediatr Transplant 2003; 7(1):18-24.

4. Stevens L A, Coresh J, Greene T, Levey A S. Assessing kidney function—measured and estimated glomerular filtration rate. N Engl J Med 2006; 354(23):2473-2483.

5. Edelstein C L. Biomarkers in kidney disease. 1st ed. Amsterdam; Boston: Academic Press/Elsevier, 2011.

6. Kaddurah-Daouk R, Kristal B S, Weinshilboum R M. Metabolomics: a global biochemical approach to drug response and disease. Annu Rev Pharmacol Toxicol 2008; 48:653-683.

7. Nicholson J K, Connelly J, Lindon J C, Holmes E. Metabonomics: a platform for studying drug toxicity and gene function. Nat Rev Drug Discov 2002; 1(2):153-161.

8. Vinayavekhin N, Homan E A, Saghatelian A. Exploring disease through metabolomics. ACS Chem Biol 2010; 5(1):91-103.

9. Wishart D S. Metabolomics: The Principles and Potential Applications to Transplantation. American Journal of Transplantation 2005; 5:2814-2820.

10. Sarwal M M. Deconvoluting the ‘omics’ for organ transplantation. Curr Opin Organ Transplant 2009; 14(5):544-551.

11. Wishart D S. Metabolomics: a complementary tool in renal transplantation. Contrib Nephrol 2008; 160:76-87.

12. Serkova N F T, Klawitter J, Freise C E, Niemann C U. H-NMR-based metabolic signatures of mild and severe ischemia/reperfusion injury in rat kidney transplants. Kidney Int 2005; 67(3):1142-1151.

13. Foxall P J M G, Bending M R, Lindon J C, Nicholson J K. NMR spectroscopy as a novel approach to the monitoring of renal transplant function. Kidney Int 1993 43(1):234-245.

14. Mao Y Y B J, Chen J H, Shou Z F, He Q, Wu J Y, Chen Y, Cheng Y Y. A pilot study of GC/MS-based serum metabolic profiling of acute rejection in renal transplantation. Transpl Immunol 2008; 19(1):74-80.

15. Sreekumar A P L, Rajendiran T M, Khan A P, Cao Q, Yu J, Laxman B, Mehra R, Lonigro R J, Li Y, Nyati M K, Ahsan A, Kalyana-Sundaram S, Han B, Cao X, Byun J, Omenn G S, Ghosh D, Pennathur S, Alexander D C, Berger A, Shuster J R, Wei J T, Varambally S, Beecher C, Chinnaiyan A M Metabolomic profiles delineate potential role for sarcosine in prostate cancer progression. Nature 2009; 457:910-914.

16. Metz T O, Page J S, Baker E S, Tang K, Ding J, Shen Y et al. High Resolution Separations and Improved Ion Production and Transmission in Metabolomics. Trends Analyt Chem 2008; 27(3):205-214.

17. Metz T O, Zhang Q, Page J S, Shen Y, Callister S J, Jacobs J M et al. The future of liquid chromatography-mass spectrometry (LC-MS) in metabolic profiling and metabolomic studies for biomarker discovery. Biomark Med 2007; 1(1):159-185.

18. Phapale P B K S, Lee H W, Lim M, Kale D D, Kim Y L, Cho J H, Hwang D, Yoon Y R. An integrative approach for identifying a metabolic phenotype predictive of individualized pharmacokinetics of tacrolimus. Clin Pharmacol Ther 2010; 87(4):426-436.

19. Gika H G, Theodoridis G A, Wingate J E, Wilson I D. Within-day reproducibility of an HPLC-MS-based method for metabonomic analysis: application to human urine. J Proteome Res 2007; 6(8):3291-3303.

20. Want E J, Nordstrom A, Morita H, Siuzdak G. From exogenous to endogenous: the inevitable imprint of mass spectrometry in metabolomics. J Proteome Res 2007; 6(2):459-468.

21. Colin A. Smith E J W, Grace O'Maille, Ruben Abagyan, and Gary Siuzdak. XCMS: Processing Mass Spectrometry Data for Metabolite Profiling Using Nonlinear Peak Alignment, Matching, and Identification Anal Chem 2006; 78(3): 779-787.

22. Bolstad B. M. R A I, M. Åstrand and T. P. Speed. A comparison of normalization methods for high density oligonucleotide array data based on variance and bias. Bioinformatics 2002; 19(2):185-193

23. Burnham A J, MacGregor J F, Viveros R. Latent variable multivariate regression modeling. Chemometrics and Intelligent Laboratory Systems 1999; 48:167-180.

24. Svante Wold J T, Anders Berglund and Henrik Antti. Some recent developments in PLS modeling Chemometrics and Intelligent Laboratory Systems 2001; 58(2):131-150.

25. al DSWe. HMDB: a knowledgebase for the human metabolome Nucleic Acids Research. Nucleic Acids Res 2007; 35:D521-D526.

26. Smith C A M, Grace O'; Want, Elizabeth J; Qin, Chuan; Trauger, Sunia A; Brandon, Theodore R; Custodio, Darlene E; Abagyan, Ruben; Siuzdak, Gary. METLIN A Metabolite Mass Spectral Database. Ther Drug Monit 2005; 27(6): 747-751.

27. Foubister V. Lipid maps project launched. J Proteome Res 2003; 2(3):576.

28. Goto MKaS. KEGG: Kyoto Encyclopedia of Genes and Genomes. Nucleic Acids Res 2000; 28(1):27-30.

29. Ron Caspi H F, Carol A. Fulcher, Pallavi Kaipa, Markus Krummenacker, Mario Latendresse, Suzanne Paley, Seung Y. Rhee, Alexander G. Shearer, Christophe Tissier, Thomas C. Walk, Peifen Zhang, and Peter D. Karp. The MetaCyc Database of metabolic pathways and enzymes and the BioCyc collection of Pathway/Genome Databases. Nucleic Acids Res 2008; 36:D623-D631.

30. Isabelle Dufort P S, Lucille Lacoste and Van Luu-The. Comparative biosynthetic pathway of androstenol and androgens. The Journal of Steroid Biochemistry and Molecular Biology 2001; 77(4-5):223-227.

31. Herget-Rosenthal S B A, Hofmann W. How to estimate GFR-serum creatinine, serum cystatin C or equations? Clin Biochem 2007; 40(3-4):153-161.

32. Fredrik Lindgren B H, Walter Karcher, Michael Sjöström, Lennart Eriksson. Model validation by permutation tests: Applications to variable selection. Journal of Chemometrics 1998; 10(5-6):521-532.

33. Wyss M K-D R. Creatine and creatinine metabolism. Physiol Rev 2000; 80(3):1107-1213.

34. Wishart D S. Metabolomics in monitoring kidney transplants. Curr Opin Nephrol Hypertens 15 2006; 15:637-642.

35. Cernácek P B H, Gerová Z, Válek A, Spustová V. Plasma tryptophan level in chronic renal failure. Clin Nephrol 1980; 14(5):246-249.

36. Niwa T T N, H. Y. RNA metabolism in uremic patients: accumulation of modified ribonucleosides in uremic serum. Kidney Int 1998; 53:1801-1806.

37. Chan W W M, Kopple J D, Swendseid M E. Citrulline Levels and Urea Cycle Enzymes in Uremic Rats. J Nutr 1974; 104(6):678-683.

38. Freedman B I H P, Sale M M, Pierson E D, Langefeld C D, Rich S S, Xu J, McDonough C, Janssen B, Yard B A, van der Woude F J, Bowden D W. A leucine repeat in the carnosinase gene CNDP1 is associated with diabetic end-stage renal disease in European Americans. Nephrol Dial Transplant 2007; 22(4):1131-1135.

39. Reynaud C B C, Boullanger P, Grange J, Barbesti S, Niveleau A. Monitoring of urinary excretion of modified nucleosides in cancer patients using a set of six monoclonal antibodies. Cancer Lett 1992; 61(3):255-262.

40. Lindon J C, Holmes E, Bollard M E, Stanley E G, Nicholson J K. Metabonomics technologies and their applications in physiological monitoring, drug safety assessment and disease diagnosis. Biomarkers 2004; 9(1):1-31.

41. Chuang H Y, Lee E, Liu Y T, Lee D, Ideker T. Network-based classification of breast cancer metastasis. Mol Syst Biol 2007; 3:140.

42. Sadeghi M, Landou I, Daniel V, Schnitzler P, Fusch G, Schefold J C et al. Strong association of phenylalanine and tryptophan metabolites with activated cytomegalovirus infection in kidney transplant recipients. Human immunology 2012; 73(2):186-192.

43. Watanabe M S M, Qureshi A R, Garcia-Lopez E, Bárány P, Heimbürger O, Stenvinkel P, Lindholm B. Consequences of low plasma histidine in chronic kidney disease patients: associations with inflammation, oxidative stress, and mortality. Am J Clin Nutr 2008; 87(6):1860-1866.

44. Fang W, Huang C C, Chu N S, Chen C J, Lu C S, Wang C C. Childhood-onset autosomal-dominant limb-girdle muscular dystrophy with cardiac conduction block. Muscle & nerve 1997; 20(3):286-292.

45. Park S W, Kim M, Brown K M, D'Agati V D, Lee H T Inhibition of sphingosine 1-phosphate receptor 2 protects against renal ischemia-reperfusion injury. J Am Soc Nephrol 2012; 23(2):266-280.

46. Janssen B, Hohenadel D, Brinkkoetter P, Peters V, Rind N, Fischer C et al. Carnosine as a protective factor in diabetic nephropathy: association with a leucine repeat of the carnosinase gene CNDP1. Diabetes 2005; 54(8):2320-2327.

47. Stevens L A, Padala S, Levey A S. Advances in glomerular filtration rate-estimating equations. Current opinion in nephrology and hypertension 2010; 19(3):298-307.

48. O. N. Steroid analysis for medical diagnosis. J Chromatogr A 2001; 935(1-2):267-278.

49. Turnell D C C J. Rapid assay for amino acids in serum or urine by pre-column derivatization and reversed-phase liquid chromatography. Clin Chem 1982; 28(3):527-531.

50. Girish B, Chheda, A. M. & Grace, J. T. Nucleosides in human urine I: isolation and identification of N2-dimethylguanosine, 1-methylinosine, and N2-methylguanosine from normal human urine. J Pharmaceut Sci 1968; 58:75-78.

51. Martens HMaM. Modified Jack-knife estimation of parameter uncertainty in bilinear modeling by partial least squares regression (PLSR). Food Quality and Preference 2000; 11(1-2):5-16.

What is claimed is:

1. A method for predicting glomerular filtration rate (GFR) from urine samples after kidney transplantation to provide an information needed for predict renal function after the transplantation, comprising detecting metabolic profiles of 5a-androst-3-en-17-one (AS), glycocholic acid (GC), sphingosine (SG), tryptophan (TR) and histidine (HT) from urine samples of patients.

2. The method for predicting glomerular filtration rate (GFR) according to claim 1, which comprises the following steps:
(1) measuring peak intensities of 5a-androst-3-en-17-one (AS), glycocholic acid (GC), sphingosine (SG), tryptophan (TR) and histidine (HT) by using LC/MS analysis from urine samples of patients;
(2) obtaining each normalized metabolite intensities from the peak intensities by performing log₂ normalization using quantile normalization algorithm;
(3) calculating predicted GFR (GFR_{Predicted}) by inserting the normalized metabolite intensities as AS, GC, SG, TR and HT in the following equation 1.
GFR_{Predicted}=0.45HT+0.46AS+0.39TR+0.54SG+0.38GC [Equation 1]

3. The method for predicting glomerular filtration rate (GFR) according to claim 2, wherein the equation can predict early and long-term GFR after kidney transplantation up to 2 years.

4. A program storage device readable by a computer, embodying a program for predicting glomerular filtration rate (GFR) from urine samples after kidney transplantation to provide an information needed for predict renal function after the transplantation, the program comprising the following steps:
(1) being provided peak intensities of 5a-androst-3-en-17-one (AS), glycocholic acid (GC), sphingosine (SG), tryptophan (TR) and histidine (HT) resulted from LC/MS analysis of urine samples of patients;
(2) obtaining each normalized metabolite intensities from the peak intensities by performing log₂ normalization using quantile normalization algorithm;
(3) calculating predicted GFR (GFR_{Predicted}) by inserting the normalized metabolite intensities as AS, GC, SG, TR and HT in the following equation 1.
GFR_{Predicted}=0.45HT+0.46AS+0.39TR+0.54SG+0.38GC [Equation 1]
